(19) United States
(12) Patent Application Publication
(10) Pub. No.: US 2004/0097557 A1
(43) Pub. Date: May 20, 2004

(54) Cyanothiophene derivatives, compositions containing such compounds and methods of use

(76) Inventors: Joseph L. Duffy, Cranford, NJ (US); Rui Liang, East Brunswick, NJ (US); Elizabeth Louise Campbell, North Brunswick, NJ (US); Sajjad A. Qureshi, Westfield, NJ (US); Bei B. Zhang, Edison, NJ (US); James R. Tata, Westfield, NJ (US); Brian A. Kirk, Basking Ridge, NJ (US); Kevin T. Chapman, Scotch Plains, NJ (US); Zenon Konteatis, Chatham Township, NJ (US)

Correspondence Address: Richard C. Billups, Merck & Co., Inc., Patent Dept., RY60-30, P. O. Box 2000, Rahway, NJ, 07065-0907, US

(21) Appl. No.: 10/706,695

(22) Filed: Nov. 12, 2003

Related U.S. Application Data
(60) Provisional application No. 60/425,795, filed on Nov. 13, 2002.

(51) Int. Cl. A61K 31/4436; A61K 31/4025; A61K 31/381

(52) U.S. Cl. 514/342; 514/422; 514/447

(57) ABSTRACT

The present invention addresses substituted cyanothiophene derivatives of the formula I:

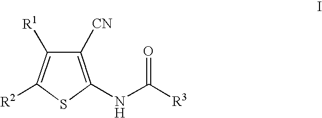

as well as compositions containing such compounds and methods of treatment. The compounds in the present invention are glucagon antagonists. The compounds block the action of glucagon at its receptor and thereby decrease the levels of plasma glucose providing a treatment of diabetes.

### CROSS-REFERENCE TO RELATED APPLICATIONS

The present invention is related to U.S. provisional application Serial No. 60/425,795, filed Nov. 13, 2002, the contents of which are hereby incorporated by reference.

### BACKGROUND OF THE INVENTION

The present invention relates to cyanothiophene compounds, methods of treatment for type 2 diabetes mellitus using substituted cyanothiophene derivatives and compositions containing such compounds.

Diabetes refers to a disease process derived from multiple causative factors and is characterized by elevated levels of plasma glucose (hyperglycemia) in the fasting state or following glucose administration during an oral glucose tolerance test. Frank diabetes mellitus (e.g., a blood glucose level≧126 mg/dL in a fasting state) is associated with increased and premature cardiovascular morbidity and mortality, and is related directly and indirectly to various metabolic conditions, including alterations of lipid, lipoprotein and apolipoprotein metabolism.

Patients with non-insulin dependent diabetes mellitus (type 2 diabetes mellitus), approximately 95% of patients with diabetes mellitus, frequently display elevated levels of serum lipids, such as cholesterol and triglycerides, and have poor blood-lipid profiles, with high levels of LDL-cholesterol and low levels of HDL-cholesterol. Those suffering from Type 2 diabetes mellitus are thus at an increased risk of developing macrovascular and microvascular complications, including coronary heart disease, stroke, peripheral vascular disease, hypertension (for example, blood pressure≧130/80 mmHg in a resting state), nephropathy, neuropathy and retinopathy.

Patients having type 2 diabetes mellitus characteristically exhibit elevated plasma insulin levels compared with nondiabetic patients; these patients have developed a resistance to insulin stimulation of glucose and lipid metabolism in the main insulin-sensitive tissues (muscle, liver and adipose tissues). Thus, Type 2 diabetes, at least early in the natural progression of the disease is characterized primarily by insulin resistance rather than by a decrease in insulin production, resulting in insufficient uptake, oxidation and storage of glucose in muscle, inadequate repression of lipolysis in adipose tissue, and excess glucose production and secretion by the liver. The net effect of decreased sensitivity to insulin is high levels of insulin circulating in the blood without appropriate reduction in plasma glucose (hyperglycemia). Hyperinsulinemia is a risk factor for developing hypertension and may also contribute to vascular disease.

Glucagon serves as the major regulatory hormone attenuating the effect of insulin in its inhibition of liver gluconeogenesis and is normally secreted by α-cells in pancreatic islets in response to falling blood glucose levels. The hormone binds to specific receptors in liver cells that triggers glycogenolysis and an increase in gluconeogenesis through cAMP-mediated events. These responses generate glucose (e.g. hepatic glucose production) to help maintain euglycemia by preventing blood glucose levels from falling significantly.

In addition to elevated levels of circulating insulin, type II diabetics have elevated levels of plasma glucagon and increased rates of hepatic glucose production. Antagonists of glucagon are useful in improving insulin responsiveness in the liver, decreasing the rate of gluconeogenesis and lowering the rate of hepatic glucose output resulting in a decrease in the levels of plasma glucose.

### SUMMARY OF THE INVENTION

A method of treating type 2 diabetes mellitus in a mammalian patient in need of such treatment is disclosed, comprising administering to the patient an anti-diabetic effective amount of a compound represented by formula I:

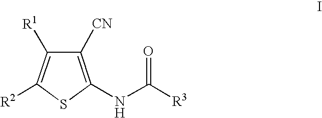

or a pharmaceutically acceptable salt or solvate thereof wherein:

R¹ is selected from the group consisting of: H, C₁₋₁₀alkyl, Aryl, Heteroaryl and Heterocyclyl,

said alkyl, Aryl, Heteroaryl and Heterocyclyl being optionally substituted with one to four substituents independently selected from R⁶;

R² is selected from the group consisting of: H, C₁₋₁₀ alkyl, C(O)C₁₋₁₀ alkyl, C(O)Aryl, C(O)Heteroaryl, C(O)Heterocyclyl CO₂R⁴ and C(O)NR⁴R⁵,

the alkyl, Aryl, Heteroaryl and Heterocyclyl portions of C(O)C₁₋₁₀alkyl, C(O)Aryl, C(O)Heteroaryl and C(O)Heterocyclyl being optionally substituted with one to four substituents independently selected from R⁶;

R³ is selected from the group consisting of: C₁₋₁₀alkyl and Aryl, said alkyl and Aryl being optionally substituted with one to four substituents independently selected from R⁶;

R⁴ is selected from the group consisting of: H, C₁₋₁₀alkyl, Aryl, Heteroaryl, Heterocyclyl, said alkyl, Aryl, Heteroaryl, and Heterocyclyl being optionally substituted with one to four substituents independently selected from R⁶;

R⁵ is selected from the group consisting of: C₁₋₁₀alkyl, Aryl, Heteroaryl and Heterocyclyl, said alkyl, cycloalkyl, Aryl Heteroaryl, and Heterocyclyl being optionally substituted with one to four substituents independently selected from R⁶;

when R² represents C(O)C₁₋₁₀alkyl, each R⁶ is independently selected from the group consisting of: halo, Aryl, Heteroaryl, Heterocyclyl, OR⁷, SR⁷, S(O)ₘR⁸, S(O)₂OR⁸, S(O)ₘNR⁷R⁸, NO₂, NR⁷R⁸, O(CR⁹R¹⁰)ₙNR⁷R⁸, C(O)R⁸, CO₂R⁷, CO₂(CR⁹R¹⁰)ₙCONR⁷R⁸, OC(O)R⁸, CN, C(O)NR⁷R⁸, NR⁷C(O)R⁸, OC(O)NR⁷R⁸, NR⁷C(O)OR⁸, NR⁷C(O)NR⁸R⁹, CR⁷(NOR⁸), (CR⁹R¹⁰)ₙ-Aryl, (CR⁹R¹⁰)ₙ-Heteroaryl, (CR⁹R¹⁰)ₙ-Heterocyclyl, CF₃ and OCF₃,

and when R² is C(O)Aryl, C(O)Heteroaryl or C(O)Heterocyclyl, and when R⁶ is a substituent on R³, R⁴ and R⁵, each R⁶ is independently selected from the group consisting of halo, C₁₋₇alkyl, Aryl, Heteroaryl, Heterocyclyl, OR⁷, SR⁷, S(O)ₘR⁸, S(O)₂OR⁸, S(O)ₘNR⁷R⁸, NO₂, NR⁷R⁸, O(CR⁹R¹⁰)ₙNR⁷R⁸, C(O)R⁸, CO₂R⁷, CO₂(CR9R¹⁰)ₙCONR⁷R⁸, OC(O)R⁸, CN, C(O)NR⁷R⁸, NR⁷C(O)R⁸, OC(O)NR⁷R⁸, NR⁷C(O)OR⁸, NR⁷C(O)NR⁸R⁹, CR⁷(NOR⁸), (CR⁹R¹⁰)ₙ-Aryl, (CR⁹R¹⁰)ₙ-Heteroaryl, (CR⁹R¹⁰)ₙ-Heterocyclyl, CF₃ and OCF₃;

wherein m is 0, 1 or 2 and n is an integer from 1 to 7, and the alkyl, Heterocyclyl, Aryl and Heteroaryl groups and portions are optionally substituted with 1-4 substituents selected from a group independently selected from R¹¹;

R⁷, R⁹ and R¹⁰ are independently selected from the group consisting of: H, C₁₋₇alkyl, Aryl, Ar-C₁₋₁₀alkyl and mono-, di- and tri- halo substituted Ar-C₁₋₁₀alkyl,

or one R⁹ and one R¹⁰ are taken together with the atoms to which they are attached and any intervening atoms and represent a ring of 3 to 8 members containing 0-2 heteroatoms independently selected from O, S and N;

R⁸ is selected from the group consisting of: C₁₋₁₀ alkyl, Aryl and C₁₋₁₀alkyl-Aryl; and

R¹¹ s selected from the group consisting of: halo, CN, C₁₋₄alkyl, Aryl, CF₃ and OH.

### DETAILED DESCRIPTION OF THE INVENTION

The invention is described herein in detail using the terms defined below unless otherwise specified.

“Alkyl”, as well as other groups having the prefix “alk”, such as alkoxy, alkanoyl and the like, refers to carbon containing groups that are linear, branched or cyclic, and combinations thereof, containing the indicated number of carbon atoms. If no number is specified, 1-10 carbon atoms are intended for linear or branched alkyl groups, and 3-10 carbon atoms are intended for cycloalkyl. When a C₁₋₁₀alkyl group is specified, this includes cycloalkyl groups containing 3-10 atoms. Cycloalkyl is thus a subset of alkyl containing 1-3 carbocyclic rings that are fused. Examples of alkyl groups include methyl, ethyl, propyl, isopropyl, butyl, sec- and tert-butyl, pentyl, hexyl, heptyl, octyl, nonyl and the like “Cycloalkyl” as used herein also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl and the like.

“Aryl” (Ar) means mono- and bicyclic aromatic rings containing 6-10 carbon atoms. Examples of aryl include phenyl, naphthyl, indenyl and the like. Ar-C₁₋₁₀alkyl refers to an aryl group attached to an alkyl group at any available point of attachment. Likewise, mon-, di- and tri-halo substituted aralkyl groups have the specified number of halo groups at any available point of attachment.

“Heteroaryl” (HAR) means a mono- or bicyclic aromatic ring or ring system containing at least one heteroatom selected from O, S and N, with each ring containing 5 to 6 atoms. Examples include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo(2,3-b)pyridyl, quinolyl, indolyl, isoquinolyl and the like. Heteroaryl also includes aromatic heterocyclic groups fused to heterocycles that are non-aromatic or partially aromatic, and aromatic heterocyclic groups fused to cycloalkyl rings.

“Heterocyclyl” (Hetcy) means mono- and bicyclic saturated rings and ring systems containing at least one heteroatom selected from N, S and O, each of said ring having from 3 to 10 atoms in which the point of attachment may be carbon or nitrogen. Examples of “heterocyclyl” include pyrrolidinyl, piperidinyl, piperazinyl, imidazolidinyl, 2,3-dihydrofuro(2,3-b)pyridyl, benzoxazinyl, tetrahydrohydroquinolinyl, tetrahydroisoquinolinyl, dihydroindolyl, and the like. The term also includes partially unsaturated monocyclic rings that are not aromatic, such as 2- or 4-pyridones attached through the nitrogen or N-substituted-(1H,3H)-pyrimidine-2,4-diones (N-substituted uracils). Preferred heterocyclyl groups include piperidinyl, piperazinyl and pyrrolidinyl.

“Halogen” (Halo) includes fluorine, chlorine, bromine and iodine.

One aspect of the invention relates to a method of treating type 2 diabetes mellitus in a mammalian patient in need of such treatment is disclosed, comprising administering to the patient an anti-diabetic effective amount of a compound represented by formula I:

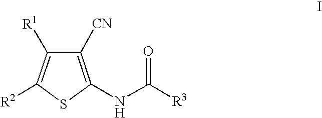

or a pharmaceutically acceptable salt or solvate thereof wherein:

R¹ is selected from the group consisting of: H, C₁₋₁₀alkyl, Aryl, Heteroaryl and Heterocyclyl,

said alkyl, Aryl, Heteroaryl and Heterocyclyl being optionally substituted with one to four substituents independently selected from R⁶;

R² is selected from the group consisting of: H, C₁₋₁₀ alkyl, C(O)C₁₋₁₀ alkyl, C(O)Aryl, C(O)Heteroaryl, C(O)Heterocyclyl CO₂R⁴ and C(O)NR⁴R⁵,

the alkyl, Aryl, Heteroaryl and Heterocyclyl portions of C(O)C₁₋₁₀alkyl, C(O)Aryl, C(O)Heteroaryl and C(O)Heterocyclyl being optionally substituted with one to four substituents independently selected from R⁶;

R³ is selected from the group consisting of: C₁₋₁₀alkyl and Aryl, said alkyl and Aryl being optionally substituted with one to four substituents independently selected from R⁶;

R⁴ is selected from the group consisting of: H, C₁₋₁₀alkyl, Aryl, Heteroaryl, Heterocyclyl, said alkyl, Aryl, Heteroaryl, and Heterocyclyl being optionally substituted with one to four substituents independently selected from R⁶;

R⁵ is selected from the group consisting of: C₁₋₁₀alkyl, Aryl, Heteroaryl and Heterocyclyl, said alkyl, cycloalkyl, Aryl Heteroaryl, and Heterocyclyl being optionally substituted with one to four substituents independently selected from R⁶;

when R² represents C(O)C₁₋₁₀alkyl, each R⁶ is independently selected from the group consisting of: halo, Aryl, Heteroaryl, Heterocyclyl, OR⁷, SR⁷, S(O)ₘR⁸, S(O)₂OR⁸, S(O)ₘNR⁷R⁸, NO₂, NR⁷R⁸, O(CR⁹R¹⁰)ₙNR⁷R⁸, C(O)R⁸, CO₂R⁷, CO₂(CR⁹R¹⁰)ₙCONR⁷R⁸, OC(O)R⁸, CN, C(O)NR⁷R⁸, NR⁷C(O)R⁸, OC(O)NR⁷R⁸, NR⁷C(O)OR⁸, NR⁷C(O)NR⁸R⁹, CR⁷(NOR⁸), (CR⁹R¹⁰)ₙ-Aryl, (CR⁹R¹⁰)ₙ-Heteroaryl, (CR⁹R¹⁰)ₙ-Heterocyclyl, CF₃ and OCF₃,

and when R² is C(O)Aryl, C(O)Heteroaryl or C(O)Heterocyclyl, and when R⁶ is a substituent on R³, R⁴ and R⁵, each R⁶ is independently selected from the group consisting of halo, C₁₋₇alkyl, Aryl, Heteroaryl, Heterocyclyl, OR⁷, SR⁷, S(O)ₘR⁸, S(O)₂OR⁸, S(O)ₘNR⁷R⁸, NO₂, NR⁷R⁸, O(CR⁹R10)ₙNR⁷R⁸, C(O)R⁸, CO₂R⁷, CO₂(CR⁹R10)ₙCONR⁷R⁸, OC(O)R⁸, CN, C(O)NR⁷R⁸, NR⁷C(O)R⁸, OC(O)NR⁷R⁸, NR⁷C(O)OR⁸, NR⁷C(O)NR⁸R⁹, CR⁷(NOR⁸), (CR⁹R¹⁰)ₙ-Aryl, (CR⁹R¹⁰)ₙ-Heteroaryl, (CR⁹R¹⁰)ₙ-Heterocyclyl, CF₃ and OCF₃;

wherein m is 0, 1 or 2 and n is an integer from 1 to 7, and the alkyl, Heterocyclyl, Aryl and Heteroaryl groups and portions are optionally substituted with 1-4 substituents selected from a group independently selected from R¹¹;

R⁷, R⁹ and R¹⁰ are independently selected from the group consisting of: H, C₁₋₇alkyl, Aryl, Ar-C₁₋₁₀alkyl and mono-, di- and tri- halo substituted Ar-C₁₋₁₀alkyl,

or one R⁹ and one R¹⁰ are taken together with the atoms to which they are attached and any intervening atoms and represent a ring of 3 to 8 members containing 0-2 heteroatoms independently selected from O, S and N;

R⁸ is selected from the group consisting of: C₁₋₁₀alkyl, Aryl and C₁₋₁₀alkyl-Aryl; and

R¹¹ is selected from the group consisting of: halo, CN, C₁₋₄alkyl, Aryl, CF₃ and OH.

In an aspect of the invention that is of particular interest, a method of treating type 2 diabetes is disclosed wherein the compound administered is a compound of formula I or a pharmaceutically acceptable salt or solvate thereof wherein R¹ represents C₁₋₁₀alkyl, preferably C₁₋₄alkyl and more preferably methyl. Within this aspect of the invention, all other variables are as originally defined.

Another aspect of the invention that is of particular interest relates to a method of treating type 2 diabetes mellitus in a mammalian patient in need of such treatment comprising administering to the patient an anti-diabetic effective amount of a compound of formula I wherein R² is selected from the group consisting of: C(O)C₁₋₁₀ alkyl, C(O)Aryl, C(O)Heteroaryl, C(O)Heterocyclyl, CO₂R⁴ and C(O)NR⁴R⁵,

the alkyl, Aryl, Heteroaryl and Heterocyclyl portions of C(O)C₁₋₁₀alkyl, C(O)Aryl, C(O)Heteroaryl and C(O)Heterocyclyl being optionally substituted with one to four substituents independently selected from R⁶. Within this aspect of the invention, all other variables are as originally defined.

More particularly, another aspect of the invention that is of interest relates to a method of treating type 2 diabetes mellitus in a mammalian patient in need of such treatment comprising administering to the patient an anti-diabetic effective amount of a compound of formula I wherein R² is C(O)C₁₋₄alkyl, C(O)-Aryl, C(O)-Heteroaryl or C(O)-Heterocyclyl, wherein the C₁₋₄alkyl, Aryl, Heteroaryl and Heterocyclyl are optionally substituted with 1-2 groups selected from R⁶, and R⁶ is selected from the group consisting of: halo, Aryl, Heteroaryl, Heterocyclyl, OR⁷, NR⁷R⁸, CF₃ and OCF₃; and the Aryl, Heteroaryl and Heterocyclyl portions are optionally substituted with halo, C₁₋₄alkyl and CF₃.

Another aspect of the invention that is of particular interest relates to a method of treating type 2 diabetes mellitus in a mammalian patient in need of such treatment comprising administering to the patient an anti-diabetic effective amount of a compound of formula I wherein R³ is C₁₋₁₀alkyl with 0-1 R⁶ groups attached. Within this aspect of the invention, all other variables are as originally defined.

In another aspect of the invention that is of particular interest, a method of treating type 2 diabetes mellitus in a mammalian patient in need of such treatment is disclosed comprising administering to the patient an anti-diabetic effective amount of a compound of formula I wherein R⁴ is H, C₁₋₁₀alkyl or Aryl, said alkyl and Aryl groups being optijnally substituted with 1-3 R⁶ groups. Within this aspect of the invention, all other variables are as originally defined.

In another aspect of the invention that is of particular interest, a method of treating type 2 diabetes mellitus in a mammalian patient in need of such treatment is disclosed comprising administering to the patient an anti-diabetic effective amount of a compound of formula I wherein R⁵ is C₁₋₁₀alkyl having 1-2 R⁶ groups attached. Within this aspect of the invention, all other variables are as originally defined.

In another aspect of the invention that is of particular interest, a method of treating type 2 diabetes mellitus in a mammalian patient in need of such treatment is disclosed comprising administering to the patient an anti-diabetic effective amount of a compound of formula I wherein R² represents a member selected from the group consisting of: CO₂R⁴ and C(O)NR⁴R⁵. Within this aspect of the invention, all other variables are as originally defined.

In yet another aspect of the invention, a method of treating type 2 diabetes mellitus in a mammalian patient in need of such treatment is disclosed comprising administering to the patient a compound of formula I or a pharmaceutically acceptable salt or solvate thereof wherein:

R¹ represents C₁₋₁₀alkyl;

R² is selected from the group consisting of: C(O)C₁₋₁₀ alkyl, C(O)Aryl, C(O)Heteroaryl, C(O)Heterocyclyl, CO₂R⁴ and C(O)NR⁴R⁵,

the alkyl, Aryl, Heteroaryl and Heterocyclyl portions of C(O)C₁₋₁₀alkyl, C(O)Aryl, C(O)Heteroaryl and C(O)Heterocyclyl being optionally substituted with one to four substituents independently selected from R⁶;

R³ is C₁₋₁₀alkyl with 0-1 R⁶ groups attached;

R⁴ is H or C₁₋₁₀alkyl optionally substituted with 1-2 R⁶ groups;

R⁵ is C₁₋₁₀alkyl having 1-2 R⁶ groups attached;

R⁶ is independently selected from the group consisting of halo, C₁₋₇alkyl, Aryl, Heteroaryl, Heterocyclyl, OR⁷, CN, (CR⁹R¹⁰)ₙ-Aryl, (CR⁹R¹⁰)ₙ-Heteroaryl, (CR⁹R¹⁰)ₙ-Heterocyclyl, CF₃ and OCF₃;

wherein n is an integer from 1 to 3, and the alkyl, Aryl, Heteroaryl and Heterocyclyl groups and portions are optionally substituted with 1-2 substituents selected from a group independently selected from R¹¹;

R⁷, R⁹ and R¹⁰ are independently selected from the group consisting of: H, C₁₋₇alkyl, Ar-C₁₋₁₀alkyl and mono-, di- and tri- halo substituted Ar-C₁₋₁₀alkyl, and

R¹¹ is selected from the group consisting of: halo, CN, C₁₋₄alkyl, Aryl, CF₃ and OH.

In yet another aspect of the invention that is of particular interest, a method of treating type 2 diabetes mellitus in a mammalian patient in need of such treatment is disclosed comprising administering to the patient an anti-diabetic effective amount of a compound of formula I wherein:

R¹ represents methyl;

R³ represents C₁₋₁₀alkyl, and R² is selected from the table below:

R²

| | | |
|---|---|---|
| CH₃ | CO₂Et | CO₂-t-Bu |
| 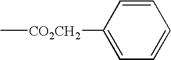 | 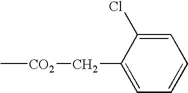 | 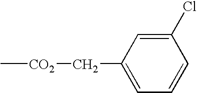 |
| 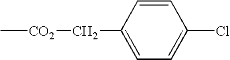 | 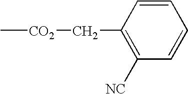 | 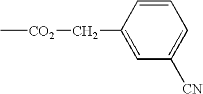 |
| 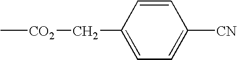 | 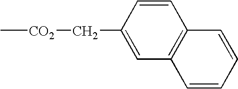 | 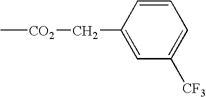 |
| 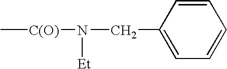 | 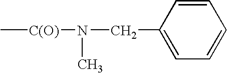 | 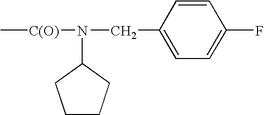 |
| —C(O)N(CH₃)₂ | 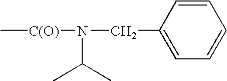 | 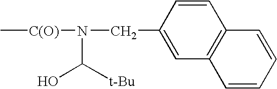 |
| 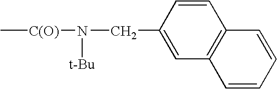 | 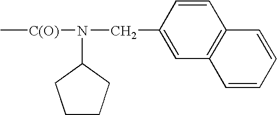 | 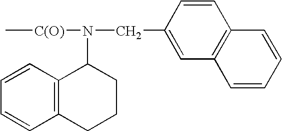 |
| 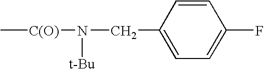 | 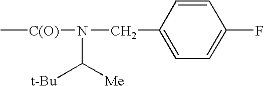 | 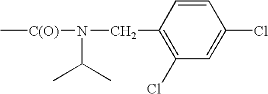 |
| 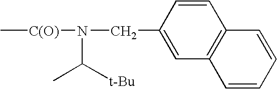 | 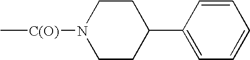 | |

as well as the pharmaceutically acceptable salts and solvates thereof.

Species within the scope of the present invention that are of particular interest include the following:

N-(3-cyano-4,5-dimethylthien-2-yl)cyclohexanecarboxamide;

isopropyl 4-cyano-5-[(2-ethylbutanoyl)amino]-3-methylthiophene-2-carboxylate;

tert-butyl 4-cyano-5-[(2-ethylbutanoyl)amino]-3-methylthiophene-2-carboxylate;

tert-butyl 4-cyano-5-[(cyclobutylcarbonyl)amino]-3-methylthiophene-2-carboxylate;

tert-butyl 4-cyano-5-[(cyclopentylcarbonyl)amino]-3-methylthiophene-2-carboxylate;

tert-butyl 4-cyano-5-[(cyclohexylcarbonyl)amino]-3-methylthiophene-2-carboxylate;

tert-butyl 4-cyano-5-(isobutyrylamino)-3-methylthiophene-2-carboxylate;

tert-butyl 4-cyano-5-[(2,2-dimethylpropanoyl)amino]-3-methylthiophene-2-carboxylate;

benzyl 4-cyano-5-[(2-ethylbutanoyl)amino]-3-methylthiophene-2-carboxylate;

2-chlorobenzyl 4-cyano-5-[(2-ethylbutanoyl)amino]-3-methylthiophene-2-carboxylate;

3-chlorobenzyl 4-cyano-5-[(2-ethylbutanoyl)amino]-3-methylthiophene-2-carboxylate;

4-chlorobenzyl 4-cyano-5-[(2-ethylbutanoyl)amino]-3-methylthiophene-2-carboxylate;

2-cyanobenzyl 4-cyano-5-[(2-ethylbutanoyl)amino]-3-methylthiophene-2-carboxylate;

3-cyanobenzyl 4-cyano-5-[(2-ethylbutanoyl)amino]-3-methylthiophene-2-carboxylate;

4-cyanobenzyl 4-cyano-5-[(2-ethylbutanoyl)amino]-3-methylthiophene-2-carboxylate;

2-naphthylmethyl 4-cyano-5-[(2-ethylbutanoyl)amino]-3-methylthiophene-2-carboxylate;

3-(trifluoromethyl)benzyl 4-cyano-5-[(2-ethylbutanoyl)amino]-3-methylthiophene-2-carboxylate;

N-benzyl-4-cyano-N-ethyl-5-[(2-ethylbutanoyl)amino]-3-methylthiophene-2-carboxamide;

4-cyano-N-cyclopentyl-5-[(2-ethylbutanoyl)amino]-N-(4-fluorobenzyl)-3-methylthiophene-2-carboxamide;

N-benzyl-4-cyano-5-[(2-ethylbutanoyl)amino]-N,3-dimethylthiophene-2-carboxamide;

4-cyano-5-[(2-ethylbutanoyl)amino]-N,N,3-trimethylthiophene-2-carboxamide;

N-benzyl-4-cyano-5-[(2-ethylbutanoyl)amino]-N-isopropyl-3-methylthiophene-2-carboxamide;

4-cyano-5-[(2-ethylbutanoyl)amino]-N-[1-(hydroxymethyl)-2,2-dimethylpropyl]-3-methyl-N-(2-naphthylmethyl)thiophene-2-carboxamide;

N-(tert-butyl)-4-cyano-5-[(2-ethylbutanoyl)amino]-3-methyl-N-(2-naphthylmethyl)thiophene-2-carboxamide;

4-cyano-5-[(2-ethylbutanoyl)amino]-3-methyl-N-(2-naphthylmethyl)-N-(1,2,2-trimethylpropyl)thiophene-2-carboxamide;

4-cyano-N-cyclopentyl-5-[(2-ethylbutanoyl)amino]-3-methyl-N-(2-naphthylmethyl)thiophene-2-carboxamide;

4-cyano-5-[(2-ethylbutanoyl)amino]-3-methyl-N-(2-naphthylmethyl)-N-(1,2,3,4-tetrahydronaphthalen-1-yl)thiophene-2-carboxamide;

N-(tert-butyl)-4-cyano-5-[(2-ethylbutanoyl)amino]-N-(4-fluorobenzyl)-3-methylthiophene-2-carboxamide;

4-cyano-5-[(2-ethylbutanoyl)amino]-N-(4-fluorobenzyl)-3-methyl-N-(1,2,2-trimethylpropyl)thiophene-2-carboxamide;

4-cyano-N-(2,4-dichlorobenzyl)-5-[(2-ethylbutanoyl)amino]-N-isopropyl-3-methylthiophene-2-carboxamide, and

N-{3-cyano-4-methyl-5-[(4-phenylpiperidin-1-yl)carbonyl]thien-2-yl}-2-ethylbutanamide, as well as the pharmaceutically acceptable salts and solvates of the compounds listed above.

The invention further includes a pharmaceutical composition which is comprised of a compound of formula I or a pharmaceutically acceptable salt or solvate thereof in combination with a pharmaceutically acceptable carrier.

Also included in the present invention is a process for preparing the pharmaceutical composition comprising combining a compound of formula I or a pharmaceutically acceptable salt or solvate thereof, with a pharmaceutically acceptable carrier.

Also included is a method of preventing or delaying the onset of type 2 diabetes mellitus in a mammalian patient in need thereof, comprising administering to said patient a compound of formula I in an amount that is effective to prevent or delay the onset of type 2 diabetes mellitus.

Also included in a method of treating, preventing or delaying the onset of diseases or conditions that are associated with type 2 diabetes mellitus. Examples include diseases and conditions selected from the group consisting of: dyslipidemias, such as elevated levels of cholesterol, triglycerides or low density lipoproteins (LDL), low levels of high density lipoprotein (HDL), microvascular or macrovascular changes and the sequellae of such conditions, such as coronary heart disease, stroke, peripheral vascular disease, hypertension, renal hypertension, nephropathy, neuropathy and retinopathy. The method entails administering to a type 2 diabetic patient, e.g., a human patient, an amount of a compound of formula I that is effective for treating, preventing or delaying the onset of such diseases or conditions.

Optical Isomers—Diastereomers—Geometric Isomers—Tautomers

Many of the compounds of formula I contain one or more asymmetric centers and thus occur as racemates and racemic mixtures, single enantiomers, diastereomeric mixtures and individual diastereomers. The present invention includes all such isomeric forms of the compounds, in pure form as well as in mixtures.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form known as keto-enol tautomers. The individual tautomers as well as mixture thereof are encompassed with compounds of Formula I.

Salts and Solvates

The term “pharmaceutically acceptable salts” refers to salts prepared from pharmaceutically acceptable substantially non-toxic bases or acids including inorganic or organic bases and inorganic or organic acids, as well as salts that can be converted into pharmaceutically acceptable salts. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

Solvates as used herein refers to the compound of formula I or a salt thereof, in association with a solvent, such as water. Representative examples include hydrates, hemihydrates, trihydrates and the like.

References to the compounds of formula I herein include the pharmaceutically acceptable salts and solvates.

This invention relates to a method of antagonizing or inhibiting the production or activity of glucagon, thereby reducing the rate of gluconeogenesis and glycogenolysis, and the concentration of glucose in plasma. In this aspect of the invention, the compound is administered to a mammalian patient in need of such treatment in an amount effective to antagonize or inhibit the production or activity of glucagon.

The compounds of formula I can be used in the manufacture of a medicament for the prophylactic or therapeutic treatment of disease states in mammals in which elevated levels of glucose are found. The process entails combining a compound of formula I or a pharmaceutically acceptable salt or solvate thereof with the carrier.

Dose Ranges

The term “anti-diabetic effective amount” refers to a representative dose of the compound of formula I that is suitable for treating a mammalian patient for type 2 diabetes mellitus or the related conditions described herein. The prophylactic, therapeutic and preventative dose of a compound of formula I will, of course, vary with the nature of the condition to be treated, the particular compound selected and its route of administration. It will also vary according to the age, weight and response of the individual patient. In general, the daily dose range lies within the range of from about 0.001 mg to about 100 mg per kg body weight, preferably about 0.005 mg to about 50 mg per kg, and more preferably 0.01 to 10 mg per kg, in single or divided doses. It may be necessary to use dosages outside of these limits in some cases, as determined by the skilled physician.

When intravenous or oral administration is employed, a representative dosage range is from about 0.001 mg to about 100 mg (preferably from 0.01 mg to about 10 mg) of a compound of formula I per kg of body weight per day, and more preferably, about 0.1 mg to about 10 mg of a compound of Formula I per kg of body weight per day.

Pharmaceutical Compositions

As mentioned above, the pharmaceutical composition comprises a compound of formula I and a pharmaceutically acceptable carrier. The term “composition” encompasses a product comprising the active and inert ingredient(s), (pharmaceutically acceptable excipients) that make up the carrier, as well as any product which results, directly or indirectly, from the combination, complexation or aggregation of any two or more of the ingredients, or from dissociation of one or more of the ingredients, or from other types of reactions or interactions between ingredients. Preferably the composition is comprised of a compound of formula I in an amount that is effective to treat, prevent or delay the onset of type 2 diabetes mellitus, in combination with the pharmaceutically acceptable carrier.

Any suitable route of administration may be employed for providing a mammal, especially a human with an effective dosage of a compound of the present invention. For example, oral, rectal, topical, parenteral, ocular, pulmonary, nasal, and the like may be employed. Examples of dosage forms include tablets, troches, dispersions, suspensions, solutions, capsules, creams, ointments, aerosols and the like, with oral tablets being preferred.

In preparing oral compositions, any of the usual pharmaceutical ingredients may be employed, such as, for example, water, glycols, oils, alcohols, flavoring agents, preservatives, coloring agents and the like in the case of oral liquids, e.g., suspensions, elixirs and solutions; or carriers such as starches, sugars, microcrystalline cellulose, diluents, granulating agents, lubricants, binders, disintegrating agents and the like in the case of oral solids, e.g., powders, capsules and tablets, with the solid oral preparations being preferred. Because of their ease of administration, tablets and capsules represent the most advantageous oral dosage unit forms. If desired, tablets may be coated by standard aqueous or nonaqueous techniques.

In addition to the common dosage forms set out above, the compounds of formula I may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any pharmaceutically acceptable method. Typically the method entails bringing into association the active ingredient with the carrier ingredients. In general, the compositions are prepared by uniformly and intimately admixing the active ingredient with any liquid or finely divided solid ingredients included in the carrier, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet contains from about 1 mg to about 1 g of the active ingredient and each cachet or capsule contains from about 1 to about 500 mg of the active ingredient.

The following are examples of pharmaceutical dosage forms for the compounds of Formula I:

| Injectable Suspen- | | | | | |
|---|---|---|---|---|---|
| sion (I.M.) | mg/mL | | Tablet | mg/tablet | |
| Compound of | 10 | | Compound of Formula I | 25 | |
| Formula I | | | Microcrystalline Cellulose | 415 | |
| Methylcellulose | 5.0 | | Povidone | 14.0 | |
| Tween 80 | 0.5 | | Pregelatinized Starch | 43.5 | |
| Benzyl alcohol | 9.0 | | Magnesium Stearate | 2.5 | |
| Benzalkonium | 1.0 | | Total | 500 | mg |
| chloride | | | | | |
| Water for injection | 1.0 | mL | | | |
| to make | | | | | |
| | mg/ | | | Per | |
| Capsule | capsule | | Aerosol | canister | |
| Compound of | 25 | | Compound of Formula I | 24 | mg |
| Formula I | | | Lecithin, NF Liq. Conc. | 1.2 | mg |
| Lactose Powder | 573.5 | | Trichlorofluoromethane, | 4.025 | g |
| Magnesium Stearate | 1.5 | | NF | | |
| Total | 600 | mg | Dichlorodifluoromethane, | 12.15 | g |
| | | | NF | | |

Combination Therapy

A compounds of formula I may be used in combination with other drugs that are used in the treatment/prevention/delaying the onset of type 2 diabetes mellitus, as well as the diseases and conditions associated with type 2 diabetes mellitus, for which the compounds are useful. Other drugs may be administered, by a route and in an amount commonly used therefor, contemporaneously or sequentially with the compound of formula I. When a compound of formula I is used contemporaneously with one or more other drugs, a pharmaceutical composition containing such other drugs in addition to the compound of formula I is preferred. Accordingly, the pharmaceutical compositions of the present invention also include those that also contain one or more other active ingredients, in addition to a compound of formula I. Examples of other active ingredients that may be combined with a compound of formula I, either administered separately or in the same pharmaceutical compositions, include, but are not limited to: (a) bis-guanides (e.g., buformin, metformin, phenformin), (b) PPAR agonists (e.g., troglitazone, pioglitazone, rosiglitazone), (c) insulin, (d) somatostatin, (e) α-glucosidase inhibitors (e.g., voglibose, miglitol, acarbose), and (f) insulin secretagogues (e.g., acetohexamide, carbutamide, chlorpropamide, glibornuride, gliclazide, glimerpiride, glipizide, gliquidine, glisoxepid, glyburide, glyhexamide, glypinamide, phenbutamide, tolazamide, tolbutamide, tolcyclamide, nateglinide and repaglinide).

The weight ratio of the compound of the Formula I to the second active ingredient may be varied within wide limits and depends upon the effective dose of each ingredient. Generally, an effective dose of each will be used. Thus, for example, when a compound of the formula I is combined with a PPAR agonist the weight ratio of the compound of the formula I to the PPAR agonist will generally range from about 1000:1 to about 1:1000, preferably about 200:1 to about 1:200. Combinations of a compound of the formula I and other active ingredients will generally also be within the aforementioned range, but in each case, an effective dose of each active ingredient should be used.

Throughout the instant application, the following abbreviations are used with the following meanings unless otherwise indicated:

| | |
|---|---|
| Bu = butyl | Bn = benzyl |
| BOC, Boc = t-butyloxycarbonyl | CBZ, Cbz = Benzyloxycarbonyl |
| DCC = Dicyclohexylcarbodiimide | DCM = dichloromethane |
| DIPEA = diisopropylethylamine | DMF = N,N-dimethylformamide |
| DMAP = 4-Dimethylaminopyridine | Et = ethyl |
| EtOAc = ethyl acetate | EtOH = ethanol |
| eq. = equivalent(s) | FAB-mass spectrum = Fast atom |
| | bombardment-mass spectroscopy |
| HOAc = acetic acid | HPLC = High pressure liquid |
| | chromatography |
| HOBT, HOBt = Hydroxybenztriazole | LAH = Lithium aluminum hydride |
| Me = methyl | PBS = phosphate buffer saline |
| Ph = phenyl | TFA = Trifluoroacetic acid |
| THF = Tetrahydrofuran | TMS = Trimethylsilane |

Compounds of the present invention may be prepared according to the methodology outlined in the following Schemes. In Scheme 1, a ketone 1 is condensed with malononitrile 2 in the presence of sulfur (S₈) and a dialkylamine (such as morpholine) in ethanol according to methods described in the literature (S. Mukherjee and A. De, J. Chem. Res. 8, 295 (1994); M. S. Mahas et al. J. Chem. Soc. 1969, (1937); A. De et al. J. Het. Chem. 29, 1213 (1992)) to afford the 2-amino-3-cyano-thiophene 3. Acylation of 3 with an appropriate anhydride or acid chloride in the presence of a trialkylamine (e.g., triethylamine or N-methyl-morpholine) according to published procedures (U. Sensfuss et al. Heteroat. Chem. 9, 529 (1998)) will afford the amide 4 corresponding to the general formula I.

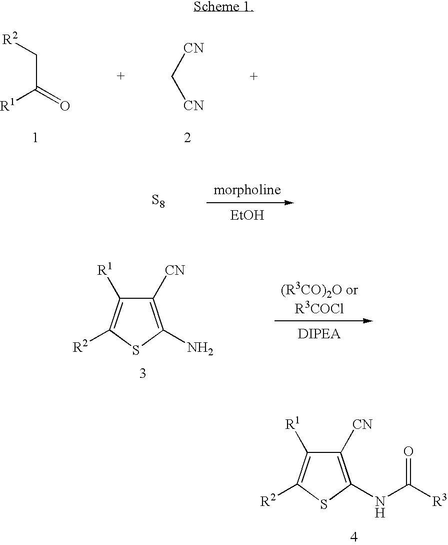

In some instances it may be necessary to carry out the thiophene synthesis in two steps, as illustrated in Scheme 2. A dicyano-alkene 5 is prepared by condensation of a ketone such as 1 and malononitrile. This intermediate is reacted with sulfur (S₈) and a dialkylamine (e.g., morpholine) in ethanol according to methods described in the literature (A. Rajca and M. Tisler, Monatch. Chem. 121, 697 (1990); B. Naumann et al., Pharmazie 53, 4 (1996)) to afford 2-amino-3-cyano-thiophene 3. Acylation of 3 with an appropriate anhydride or acid chloride in the presence of a trialkylamine (e.g., diisopropylethylamine) according to published procedures (U. Sensfuss et al. Heteroat. Chem. 9, 529 (1998)) will afford the thiopheneamide represented by formula I.

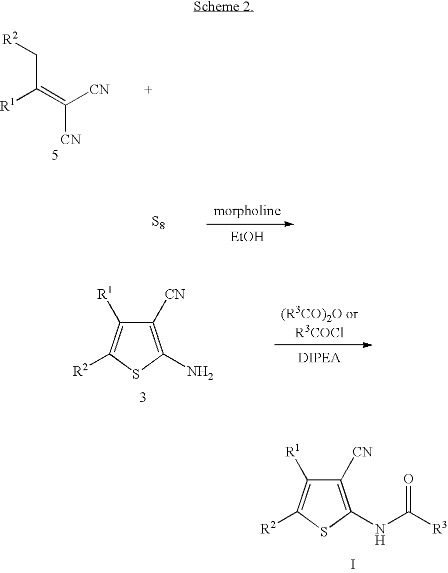

It is recognized that when the ketone 1 is not a symmetrically substituted ketone, the product 3 may be formed as a mixture of positional isomers. These isomers may be separated at any stage in the synthetic sequence by preparative thin layer chromatography, flash chromatography on silica gel as described by W. C. Still et al., J. Org. Chem., 43, 2923 (1978), or HPLC. Compounds that are purified by HPLC may be isolated as the corresponding salt.

A wide variety of ketones corresponding to 1 are commercially available, known in the literature, or may be conveniently prepared by a variety of methods known to those skilled in the art. One such example of a ketone that may be used in the synthesis of compounds of the general formula I is tert-butyl 3-oxoalkanoate 6 in Scheme 3. The intermediate 7 is obtained as illustrated in Scheme 1, followed by acylation to afford intermediate 8.

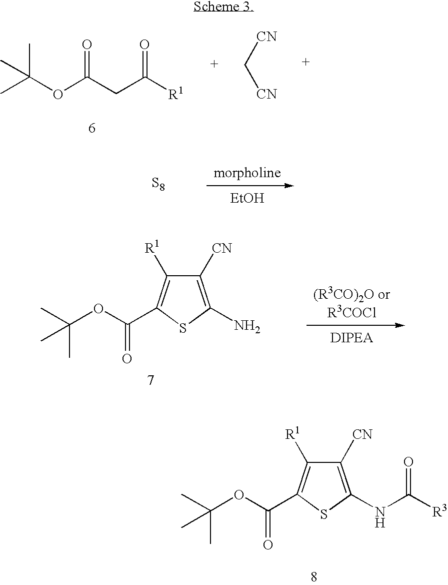

Intermediate 8 from Scheme 3 may be further manipulated to derive other compounds of the present invention. As illustrated in Scheme 4, the tert-butyl ester may be removed to reveal the carboxylate 9 using acidic conditions such as trifluoromethylacetic acid in an aprotic solvent such as dichloromethane at 0-50° C. for 3-48 h. The carboxylate intermediate 9 may be esterified to form compounds such as 10 by a variety of methods. Two such methods are also illustrated in Scheme 4. In the first such method, the free acid may be combined with an alkyl bromide in the presence of a tertiary amine base such as di-iso-propylethylamine in an organic solvent such as dichloromethane at 20-50° C. for 3-48 h, to afford the corresponding ester 10. Alternatively, the carboxylate intermediate 9 may be activated with a coupling reagent such as 2-chloro-1-methylpyridinium iodide in the presence of a tertiary amine base such as di-iso-propylethylamine in an organic solvent such as dichloromethane at 20-50° C. for 3-48 h, to afford the corresponding ester 10.

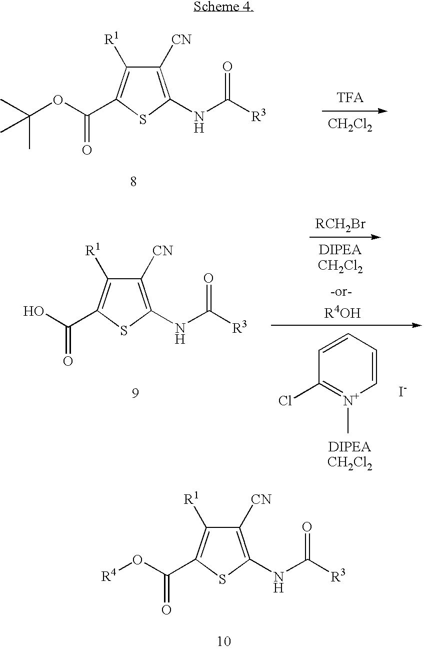

Intermediate 9 from Scheme 4 may also be manipulated to form the amide derivatives corresponding to formula 1 by coupling to a primary amine, as illustrated in Scheme 5. A variety of primary amines are commercially available, known in the literature, or may be readily prepared by those skilled in the art. The coupling of these amines to intermediate 9 may be accomplished using bromo-tris-pyrrolidino-phosphonium hexafluorophosphate (PyBrop) in the presence of an amine base such as di-iso-propylethylamine in dichloromethane at ambient temperature for 3-48 h to afford the amide product 12.

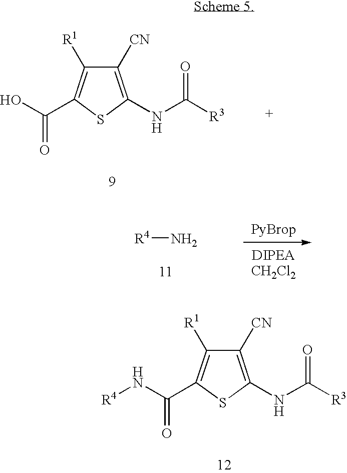

Alternatively, intermediate 9 from Scheme 4 may be coupled with a secondary amine to form the corresponding amide derivatives corresponding to formula I, as illustrated in Scheme 6. A variety of secondary amines are commercially available, known in the literature, or may be readily prepared by those skilled in the art. One such method of preparation is also illustrated in Scheme 6, where a primary amine R⁴—NH₂ may be combined with an aldehyde RCHO in dichloromethane the presence of a reducing agent such as sodium triacetoxyborohydride to form the secondary amine intermediate 13. This amine may then be coupled to the acid intermediate 9 using bromo-tris-pyrrolidino-phosphonium hexafluorophosphate (PyBrop) in the presence of an amine base such as di-iso-propylethylamine in dichloromethane at ambient temperature for 3-48 h to afford the amide product 14.

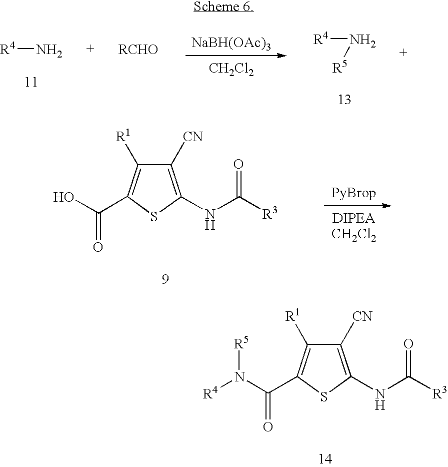

The following examples are provided so that the invention might be more fully understood. These examples are illustrative only and should not be construed as limiting the invention in any way.

The compounds listed in Table 1 are used in the present invention and are commercially available from Olivia Scientific, Inc., 475 Wall Street, Princeton, N.J. 08540.

TABLE 1

| Example | |
|---|---|
| No. | COMPOUND |
| 1 | N-(3-Cyano-4,5-dimethylthien-2-yl)cyclohexanecarboxamide |
| 2 | Isopropyl 4-cyano-5-[(2-ethylbutanoyl)amino]-3- |
| | methylthiophene-2-carboxylate |

### EXAMPLE 3

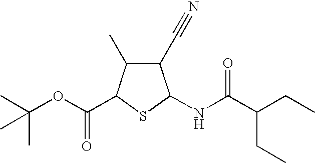

Step A. tert-Butyl 5-amino-4-cyano-3-methylthiophene-2-carboxylate. The title compound was prepared via the sequence outlined in Scheme 1. Thus to 3.32 mL (20.0 mmol) of tert-butyl 3-oxobutanoate in 50 mL of EtOH was added 1.30 mL (20.0 mmol) of malononitrile, followed by 2.62 mL (30.0 mmol) of morpholine, then 0.640 g (20.0 mmol) of elemental sulfur. The mixture was heated to 70° C. for 2 h, then cooled to ambient temperature and purified directly by flash chromatography (30% EtOAc in hexanes), affording the title compound as a beige solid. ¹H NMR (500 MHz, CDCl₃) 5.25 (s, 2H), 2.51 (s, 3H), 1.57 (s, 9H); mass spectrum (ES) m/e=183 (M+H minus tert-butyl).

Step B. tert-Butyl 4-cyano-5-[(2-ethylbutanoyl)amino]-3-methylthiophene-2-carboxylate. To 2.38 g (10.0 mmol) of the intermediate prepared in Step A in 30 mL of dichloromethane was added 1.74 mL (10.0 mmol) of di-iso-propylethylamine, followed by 1.38 mL (10.0 mmol) of 2-ethylbutanoyl chloride. After 4 h at ambient temperature, the mixture was diluted with an equal volume of saturated aqueous NaHCO₃ and extracted twice with dichloromethane. The combined organic layers were dried (Na₂SO₄) and concentrated in vacuo. Purification of 100 mg of the extract by reverse phase preparative HPLC afforded the title compound as a white solid. ¹H NMR (500 MHz, CDCl₃) 8.61 (s, 1H), 5.02 (s, 3H), 2.33 (m, 1H), 1.78 (m, 2H), 1.69 (m, 2H), 1.57 (s, 9H), 0.95 (t, J=7.5 Hz, 6H); mass spectrum (ES) m/e=337.2 (M+H).

Using the intermediate prepared in Example 3 Step A, and following the procedure outlined in example 3 step B, the compounds listed in Table 2 were prepared.

TABLE 2

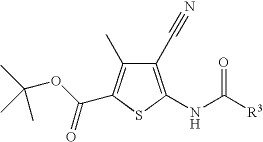

| Example | R³ | Mass spectrum (ES) m/e |
|---|---|---|
| 4 | 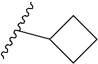 | 321.2 (M + 1) |
| 5 | 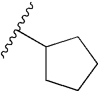 | 335.2 (M + 1) |
| 6 | 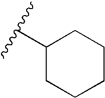 | 349.2 (M + H) |
| 7 | 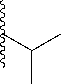 | 309.2 (M + H) |
| 8 | 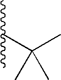 | 323.2 (M + H) |

### EXAMPLE 9

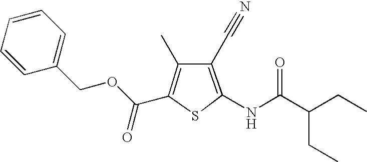

Benzyl 4-cyano-5-[(2-ethylbutanoyl)amino]-3-methylthiophene-2-carboxylate. The title compound was prepared via the sequence outlined in scheme 1 using the procedure described in example 3, starting from benzyl 3-oxobutanoate. Purification by preparative reversed phase HPLC afforded the title compound as a white solid. ¹H NMR (500 MHz, CDCl₃) 8.89 (s, 1H), 7.41 (m, 5H), 5.31 (s, 2H), 2.63 (s, 3H), 2.35 (m, 1H), 1.77 (m, 2H), 1.65 (m, 2H), 0.96 (t, J=7.5 Hz, 6H); mass spectrum (ES) m/e=371.2 (M+H).

### EXAMPLE 10

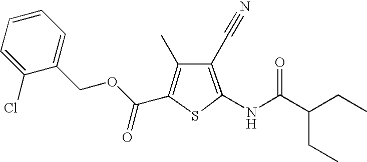

Step A. 4-Cyano-5-[(2-ethylbutanoyl)amino]-3-methylthiophene-2-carboxylic acid. The title compound from example 3 was prepared as in example 3 step B from 1.19 g (5.00 mmol) of tert-butyl 5-amino-4-cyano-3-methylthiophene-2-carboxylate, 0.740 mL (5.50 mmol) of 2-ethylbutanoyl chloride. The crude product from this reaction was dissolved in 10 mL of CH₂Cl₂, and to this solution was added 10 mL of trifluoroacetic acid. After 1 h at ambient temperature, the reaction was concentrated in vacuo, and passed through a short plug of silica, eluting with 30% EtOAc in hexane. This afforded the title compound as a white solid. ¹H NMR (500 MHz, CD₃OD) 2.63 (m, 1H), 2.58 (s, 3H), 1.69 (m, 2H), 1.60 (m, 2H), 0.94 (t, J=7.3 Hz, 6H); mass spectrum (ES) m/e=281.2 (M+H).

Step B. 2-Chlorobenzyl 4-cyano-5-[(2-ethylbutanoyl)amino]-3-methylthiophene-2-carboxylate. To a solution of 0.070 g (0.250 mmol) of the intermediate prepared in step A in 2 mL of CH₂Cl₂ was added 0.087 mL of di-iso-propylethylamine, followed by 0.032 mL (0.250 mmol) of 2-chlorobenzylbromide. After 24 h at ambient temperature, the reaction was diluted with 20 mL of CH₂Cl₂, followed by washing with 20 mL of 1 N aqueous NaOH, then 1 N aqueous HCl. The organic layer was dried (Na₂SO₄) and concentrated in vacuo. The crude material was purified by preparative reversed phase HPLC, affording the title compound as a white solid. ¹H NMR (500 MHz, CDCl₃) 8.86 (s, 1H), 7.49 (m, 1H), 7.43 (m, 1H), 7.30 (m, 2H), 5.42 (s, 2H), 2.65 (s, 3H), 2.34 (m, 1H), 1.74 (m, 2H), 1.64 (m, 2H), 0.98 (t, J=7.5 Hz, 6H); mass spectrum (ES) m/e=405.2 (M+1).

Using the intermediate prepared in example 10 step A, and following the procedure outlined in example 10 step B, the compounds listed in Table 3 were prepared.

TABLE 3

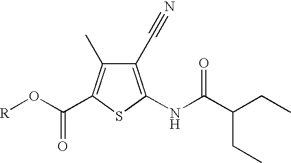

| Example | R⁴ | Mass spectrum (ES) m/e |
|---|---|---|
| 11 | 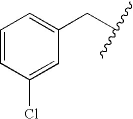 | 405.2 (M + 1) |
| 12 | 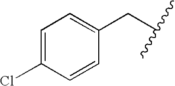 | 405.2 (M + 1) |
| 13 | 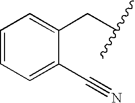 | 396.2 (M + H) |
| 14 | 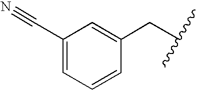 | 396.2 (M + H) |
| 15 | 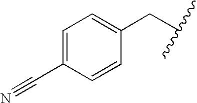 | 396.2 (M + H) |
| 16 | 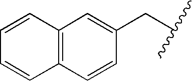 | 421.3 (M + H) |
| 17 | 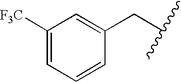 | 439.2 (M + H) |

### EXAMPLE 18

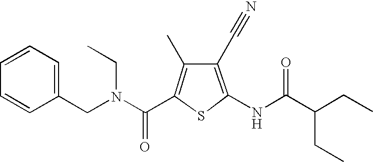

N-Benzyl-4-cyano-N-ethyl-5-[(2-ethylbutanoyl)amino]-3-methylthiophene-2-carboxamide. To a solution of 0.100 g (0.357 mmol) of the intermediate prepared in example 10 step A in 3 mL of CH₂Cl₂ was added 0.50 g (0.39 mmol) of N-benzylethylamine, followed by 0.200 g (1.10 mmol) of di-iso-propylethylamine, 0.17 g (0.10 mmol) of HOBT, and 0.070 g (0.39 mmol) of EDC. After 1 h at ambient temperature the reaction was diluted with 50 mL of CH₂Cl₂, and washed with an equal volume of 1 N NaOH, followed by an equal volume of 1 N HCl. The organic layer was dried (Na₂SO₄) and concentrated in vacuo. The crude product was purified by preparative reversed phase HPLC, affording the title compound as a white solid. ¹H NMR (500 MHz, CDCl₃) 8.96 (s, 1H), 7.31 (m, 5H), 4.70 (s, 2H), 3.41 (q, J=7.0 Hz, 2H), 2.34 (m, 1H), 2.32 (s, 3H), 1.67 (m, 2H), 1.62 (m, 2H), 1.17 (t, J=7.0 Hz, 3 H), 0.96 (t, J=7.0 Hz, 6H); mass spectrum (ES) m/e=398.3 (M+H).

### EXAMPLE 19

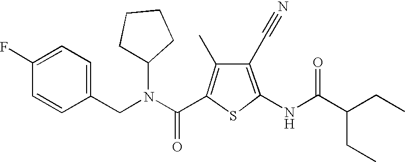

Step A. N-(4-Fluorobenzyl)cyclopentanamine. To a solution of 0.197 mL (2.00 mmol) of cyclopentylamine in 5 mL of CH₂Cl₂ was added 0.215 mL (2.00 mmol) of 4-fluorobenzaldehyde, followed by 0.650 g (3.00 mmol) of sodium triacetoxyborohydride. After 24 h at ambient temperature, the reaction was quenched with 20 mL of saturated aqueous NaHCO₃ and extracted with an equal volume of CH₂Cl₂. The combined organic layers were dried (Na₂SO₄) and concentrated in vacuo, affording the title compound as a colorless oil. This material was used without further purification.

Step B. 4-Cyano-N-cyclopentyl-5-[(2-ethylbutanoyl)amino]-N-(4-fluorobenzyl)-3-methylthiophene-2-carboxamide. The title compound was prepared using the procedure outlined in example 18, and using the amine prepared in example 19, step A, and the carboxylic acid prepared in example 10, step A. Purification by preparative reversed phase HPLC afforded the title compound as a white solid. ¹H NMR (500 MHz, CDCl₃) 8.66 (s, 1H), 7.23 (m, 2H), 7.02 (m, 2H), 4.61 (s, 2H), 4.42 (m, 1H), 2.68 (m, 3H), 2.33 (s, 3H), 1.85 (m, 2H), 1.75 (m, 2H), 1.65 (m, 3H), 1.54 (m, 1H), 0.96 (t, J=7.4 Hz, 6H); mass spectrum (ES) m/e=456.3 (M+1).

Using the intermediate prepared in example 10 step A, and following the procedure outlined in example 19, the compounds listed in Table 4 were prepared.

TABLE 4

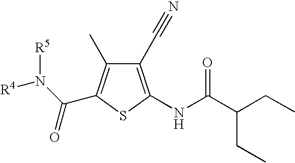

| | | | Mass |
|---|---|---|---|
| | | | spectrum (ES) |
| Example | R⁴ | R⁵ | m/e |
| 20 | 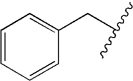 | 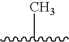 | 384.3 (M + 1) |
| 21 | 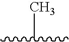 | 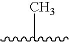 | 308.2 (M + 1) |
| 22 | 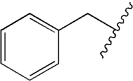 | 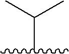 | 412.3 (M + H) |
| 23 | 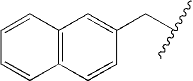 | 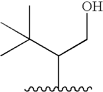 | 520.3 (M + H) |
| 24 | 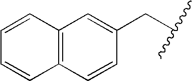 | 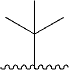 | 476.3 (M + H) |
| 25 | 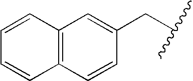 | 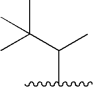 | 504.3 (M + H) |
| 26 | 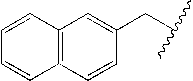 | 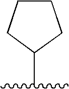 | 488.3 (M + H) |
| 27 | 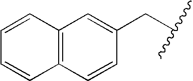 | 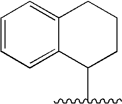 | 550.3 (M + 1) |
| 28 | 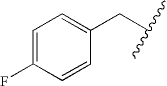 | 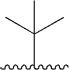 | 444.3 (M + H) |
| 29 | 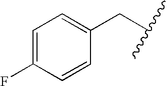 | 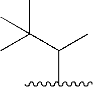 | 472.3 (M + H) |
| 30 | 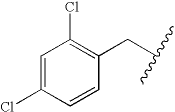 | 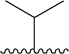 | 480.2 (M + H) |

### EXAMPLE 31

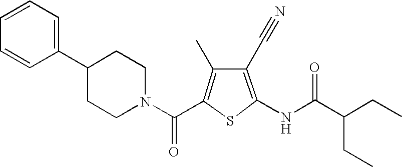

N-{3-Cyano-4-methyl-5-[(4-phenylpiperidin-1-yl)carbonyl]thien-2-yl}-2-ethylbutanamide. Using the intermediate prepared in example 10 step A, and following the procedure outlined in example 19, the title compound was prepared. ¹H NMR (500 MHz, CDCl₃) 8.86 (s, 1H), 7.34 (t, J=7.5 Hz, 2H), 7.25 (m, 3H), 4.41 (s, 1H), 3.06 (t, J=12 Hz, 2H), 2.81 (tt, J=3.5 Hz, J=12 Hz, 1H), 2.36 (s, 3H), 2.32 (m, 1H), 1.95 (d, J=12.5 Hz, 2H), 1.73 (m, 2H), 1.66 (m, 2H), 0.96 (t, J=7.5 Hz, 6H); mass spectrum (ES) m/e=424.3 (M+1).

### Biological Assays

The ability of the compounds of the present invention to inhibit the binding of glucagon and their utility in treating or preventing type 2 diabetes mellitus and the related conditions can be demonstrated by the following in vitro assays.

Glucagon Receptor Binding Assay

A stable CHO (Chinese hamster ovary) cell line expressing cloned human glucagon receptor was maintained as described (Chicchi et al. J Biol Chem 272, 7765-9(1997); Cascieri et al. J Biol Chem 274, 8694-7(1999)). To determine antagonistic binding affinity of compounds 0.002 mg of cell membranes from these cells were incubated with ¹²⁵I-Glucagon (New England Nuclear, MA) in a buffer containing 50 mM Tris-HCl (pH 7.5), 5 mM MgCl₂, 2 mM EDTA, 12% Glycerol, and 0.200 mg WGA coated PVT SPA beads (Amersham), +/−compounds or 0.001 mM unlabeled glucagon. After 4-12 hours incubation at room temperature, the radioactivity bound to the cell membranes was determined in a radioactive emission detection counter (Microbeta-Wallace). Data was analyzed using the software program Prism® from GraphPad. The IC₅₀ were calculated using non-linear regression analysis assuming single site competition.

High Throughput Screening (HTS) Protocol for Glucagon Receptor Binding Assay

Another form of the binding assay was developed suitable for high-throughput screening for modulators of receptor activity. Fully automated or semi-automated protocols and robotic and workstation instruments were utilized for the HTS assay as would be recognized by those practiced in the art. In a typical configuration of the assay, 0.002 mg of cell membrane (as described above) were preincubated with 0.200 mg of WGA-coated PVT beads in buffer containing 100 mM Tris-HCl pH 7.5, 10 mM MgCl₂, 4 mM EDTA, 24% Glycerol, and 0.2% BSA. The membrane/bead mixture was then dispensed (0.050 mL) into each well of 96-well plates (Wallac Isoplates, white clear bottom) containing 0.100 mL of test compounds or control solutions. A second addition (0.050 mL) was then dispensed into the wells of the plate containing ¹²⁵I-Glucagon (approximately 25,000 CPM). The solutions were dispensed using a Multidrop Stacker 20 (Titertek) liquid dispenser. An adhesive plate seal (Packard) was applied and the plates were shaken for 5 minutes. The plates were further incubated at ambient temperature for several hours for establishment of equilibrium (typically 5 hours) and the signal was stable for up to three days. The plates were read in a scintillation counter (Wallac Microbeta) for 1 min/well. Activity of test compounds was calculated by comparing to the total scintillation signal (CPM) of control samples with no compound and with 0.001 mM unlabeled-glucagon.

Inhibition of Glucagon-Stimulated Intracellular cAMP Formation

Exponentially growing CHO cells expressing human glucagon receptor were harvested with the aid of enzyme-free dissociation media (Specialty Media), pelleted at low speed, and re-suspended in cell suspension buffer [75 mM Tris-HCl pH7.5, 250 mM Sucrose, 25 mM MgCl₂, 1.5 mM EDTA, 0.1 mM Ro-20-1724 (Biomol, Inc.), 0.2% bovine serum albumin and one tablet of complete™ (Boehringer), which contains a cocktail of protease inhibitors, for each 50 ml of buffer]. An adenylate cyclase assay was setup using an Adenylate Cyclase Assay kit (SMP-004B) from New England Nuclear (NEN) as per manufacturer instructions. Briefly, compounds were diluted from stocks in a cell stimulation buffer supplied with the kit. Cells prepared as above were preincubated in flash plates coated with anti-cAMP antibodies (NEN) in presence of compounds or DMSO controls for 40 minutes, and then stimulated with glucagon (250 pM) for an additional 40 minutes. The cell stimulation was stopped by addition of equal amount of a detection buffer containing lysis buffer as well as ¹²⁵I-labeled cAMP tracer (NEN). After 3-6 h of incubation at room temperature the bound radioactivity was determined in a liquid scintillation counter (TopCount-Packard Instruments). Activity of test compounds was calculated by comparing to the total scintillation signal (CPM) of control samples with no compound and with 0.001 mM unlabeled-glucagon.

Certain embodiments of the invention has been described in detail; however, numerous other embodiments are contemplated as falling within the invention. Thus, the claims are not limited to the specific embodiments described herein. All patents, patent applications and publications that are cited herein are hereby incorporated by reference in their entirety.

What is claimed is:

1. A method of treating type 2 diabetes mellitus in a mammalian patient in need of such treatment, comprising administering to the patient an anti-diabetic effective amount of a compound represented by formula I: 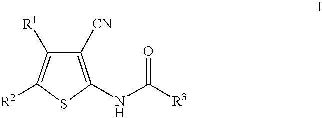
or a pharmaceutically acceptable salt or solvate thereof wherein:
R¹ is selected from the group consisting of: H, C₁₋₁₀alkyl, Aryl, Heteroaryl and Heterocyclyl,
said alkyl, Aryl, Heteroaryl and Heterocyclyl being optionally substituted with one to four substituents independently selected from R⁶;
R² is selected from the group consisting of: H, C₁₋₁₀ alkyl, C(O)C₁₋₁₀ alkyl, C(O)Aryl, C(O)Heteroaryl, C(O)Heterocyclyl CO₂R⁴ and C(O)NR⁴R⁵,
the alkyl, Aryl, Heteroaryl and Heterocyclyl portions of C(O)C₁₋₁₀alkyl, C(O)Aryl, C(O)Heteroaryl and C(O)Heterocyclyl being optionally substituted with one to four substituents independently selected from R⁶;
R³ is selected from the group consisting of: C₁₋₁₀alkyl and Aryl, said alkyl and Aryl being optionally substituted with one to four substituents independently selected from R⁶;
R⁴ is selected from the group consisting of: H, C₁₋₁₀alkyl, Aryl, Heteroaryl, Heterocyclyl, said alkyl, Aryl, Heteroaryl, and Heterocyclyl being optionally substituted with one to four substituents independently selected from R⁶;
R⁵ is selected from the group consisting of: C₁₋₁₀alkyl, Aryl, Heteroaryl and Heterocyclyl, said alkyl, cycloalkyl, Aryl Heteroaryl, and Heterocyclyl being optionally substituted with one to four substituents independently selected from R⁶;
when R² represents C(O)C₁₋₁₀alkyl, each R⁶ is independently selected from the group consisting of: halo, Aryl, Heteroaryl, Heterocyclyl, OR⁷, SR⁷, S(O)ₘR⁸, S(O)₂OR⁸, S(O)ₘNR⁷R⁸, NO₂, NR⁷R⁸, O(CR⁹R¹⁰)ₙNR⁷R⁸, C(O)R⁸, CO₂R⁷, CO₂(CR⁹R¹⁰)ₙCONR⁷R⁸, OC(O)R⁸, CN, C(O)NR⁷R⁸, NR⁷C(O)R⁸, OC(O)NR⁷R⁸, NR⁷C(O)OR⁸, NR⁷C(O)NR⁸R⁹, CR⁷(NOR⁸), (CR⁹R¹⁰)ₙ-Aryl, (CR⁹R¹⁰)ₙ-Heteroaryl, (CR⁹R¹⁰)ₙ-Heterocyclyl, CF₃ and OCF₃,
and when R² is C(O)Aryl, C(O)Heteroaryl or C(O)Heterocyclyl, and when R⁶ is a substituent on R³, R⁴ and R⁵, each R⁶ is independently selected from the group consisting of halo, C₁₋₇alkyl, Aryl, Heteroaryl, Heterocyclyl, OR⁷, SR⁷, S(O)ₘR⁸, S(O)₂OR⁸, S(O)ₘNR⁷R⁸, NO₂, NR⁷R⁸, O(CR⁹R¹⁰)ₙNR⁷R⁸, C(O)R⁸, CO₂R⁷, CO₂(CR⁹R¹⁰)ₙCONR⁷R⁸, OC(O)R⁸, CN, C(O)NR⁷R⁸, NR⁷C(O)R⁸, OC(O)NR⁷R⁸, NR⁷C(O)OR⁸, NR⁷C(O)NR⁸R⁹, CR⁷(NOR⁸), (CR⁹R¹⁰)ₙ-Aryl, (CR⁹R¹⁰)ₙ-Heteroaryl, (CR⁹R¹⁰)ₙ-Heterocyclyl, CF₃ and OCF₃;
wherein m is 0, 1 or 2 and n is an integer from 1 to 7, and the alkyl, Heterocyclyl, Aryl and Heteroaryl groups and portions are optionally substituted with 1-4 substituents selected from a group independently selected from R¹¹;
R⁷, R⁹ and R¹⁰ are independently selected from the group consisting of: H, C₁₋₇alkyl, Aryl, Ar-C₁₋₁₀alkyl and mono-, di- and tri- halo substituted Ar-C₁₋₁₀alkyl,
or one R⁹ and one R¹⁰ are taken together with the atoms to which they are attached and any intervening atoms and represent a ring of 3 to 8 members containing 0-2 heteroatoms independently selected from O, S and N;
R⁸ is selected from the group consisting of: C₁₋₁₀ alkyl, Aryl and C₁₋₁₀alkyl-Aryl; and
R¹¹ is selected from the group consisting of: halo, CN, C₁₋₄alkyl, Aryl, CF₃ and OH.

2. A method of treating type 2 diabetes in accordance with claim 1 wherein the compound administered is a compound of formula I or a pharmaceutically acceptable salt or solvate thereof wherein R¹ represents C₁₋₁₀alkyl.

3. A method of treating type 2 diabetes mellitus in accordance with claim 2 wherein R¹ represents C₁₋₄alkyl.

4. A method of treating type 2 diabetes mellitus in accordance with claim 3 wherein R¹ represents methyl.

5. A method of treating type 2 diabetes mellitus in accordance with claim 1 wherein R² is selected from the group consisting of: C(O)C₁₋₁₀ alkyl, C(O)Aryl, C(O)Heteroaryl, C(O)Heterocyclyl, CO₂R⁴ and C(O)NR⁴R⁵,
the alkyl, Aryl, Heteroaryl and Heterocyclyl portions of C(O)C₁₋₁₀alkyl, C(O)Aryl, C(O)Heteroaryl and C(O)Heterocyclyl being optionally substituted with one to four substituents independently selected from R⁶.

6. A method of treating type 2 diabetes in accordance with claim 5 wherein R² is C(O)C₁₋₄alkyl, C(O)-Aryl, C(O)-Heteroaryl or C(O)-Heterocyclyl, and the C₁₋₄alkyl, Aryl, Heteroaryl and Heterocyclyl portions are optionally substituted with 1-2 groups selected from R⁶;
and R⁶ is selected from the group consisting of: halo, Aryl, Heteroaryl, Heterocyclyl, OR⁷, NR⁷R⁸, CF₃ and OCF₃; and the Aryl, Heteroaryl and Heterocyclyl portions are optionaly substituted with halo, C₁₋₄alkyl and CF₃.

7. A method of treating type 2 diabetes in accordance with claim 1 wherein R³ is C₁₋₁₀alkyl with 0-1 R⁶ groups attached.

8. A method of treating type 2 diabetes in accordance with claim 1 wherein R⁴ is H, C₁₋₁₀alkyl or Aryl, said alkyl and Aryl groups being optionally substituted with 1-3 R⁶ groups

9. A method of treating type 2 diabetes in accordance with claim 1 wherein R⁵ is C₁₋₁₀alkyl having 1-2 R⁶ groups attached.

10. A method of treating type 2 diabetes in accordance with claim 1 wherein R² represents a member selected from the group consisting of: CO₂R⁴ and C(O)NR⁴R⁵.

11. A method of treating type 2 diabetes in accordance with claim 1 wherein:
R¹ represents C₁₋₁₀alkyl;
R² is selected from the group consisting of: C(O)C₁₋₁₀ alkyl, C(O)Aryl, C(O)Heteroaryl, C(O)Heterocyclyl, CO₂R⁴ and C(O)NR⁴R⁵,
the alkyl, Aryl, Heteroaryl and Heterocyclyl portions of C(O)C₁₋₁₀alkyl, C(O)Aryl, C(O)Heteroaryl and C(O)Heterocyclyl being optionally substituted with one to four substituents independently selected from R⁶;
R³ is C₁₋₁₀alkyl with 0-1 R⁶ groups attached;
R⁴ is H or C₁₋₁₀alkyl optionally substituted with 1-2 R⁶ groups;
R⁵ is C₁₋₁₀alkyl having 1-2 R⁶ groups attached;
R⁶ is independently selected from the group consisting of halo, C₁₋₇alkyl, Aryl, Heteroaryl, Heterocyclyl, OR⁷, CN, (CR⁹R¹⁰)ₙ-Aryl, (CR⁹R¹⁰)ₙ-Heteroaryl, (CR⁹R¹⁰)ₙ-Heterocyclyl, CF₃ and OCF₃;
wherein n is an integer from 1 to 3, and the alkyl, Aryl, Heteroaryl and Heterocyclyl groups and portions are optionally substituted with 1-2 substituents selected from a group independently selected from R¹¹;
R⁷, R⁹ and R¹⁰ are independently selected from the group consisting of: H, C₁₋₇alkyl, Ar-C₁₋₁₀alkyl and mono-, di- and tri- halo substituted Ar-C₁₋₁₀alkyl, and
R¹¹ is selected from the group consisting of: halo, CN, C₁₋₄alkyl, Aryl, CF₃ and OH.

12. A method of treating type 2 diabetes in accordance with claim 11 wherein:
R¹ represents methyl;
R³ represents C₁₋₁₀alkyl, and R² is selected from the table below:
R²

| | | |
|---|---|---|
| CH₃ | CO₂Et | CO₂-t-Bu |
| 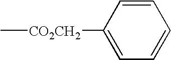 | 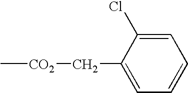 | 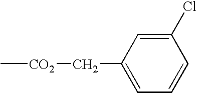 |
| 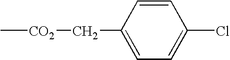 | 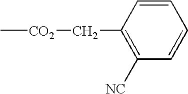 | 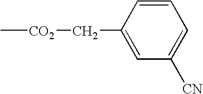 |
| 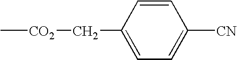 | 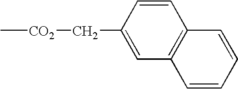 | 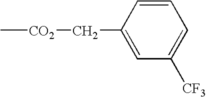 |
| 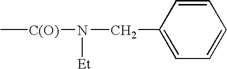 | 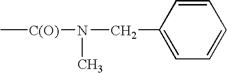 | 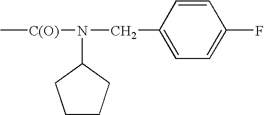 |
| —C(O)N(CH₃)₂ | 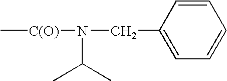 | 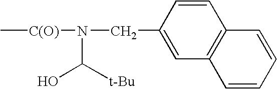 |
| 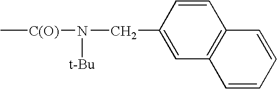 | 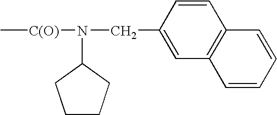 | 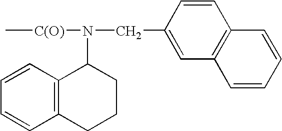 |
| 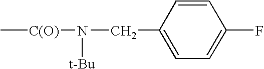 | 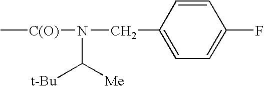 | 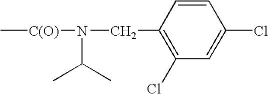 |
| 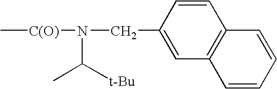 | 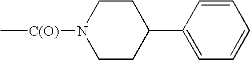 | |

13. A method of treating type 2 diabetes mellitus in accordance with claim 1 wherein the compound adminstered is selected from the group consisting of:
N-(3-cyano-4,5-dimethylthien-2-yl)cyclohexanecarboxamide;
isopropyl 4-cyano-5-[(2-ethylbutanoyl)amino]-3-methylthiophene-2-carboxylate;
tert-butyl 4-cyano-5-[(2-ethylbutanoyl)amino]-3-methylthiophene-2-carboxylate;
tert-butyl 4-cyano-5-[(cyclobutylcarbonyl)amino]-3-methylthiophene-2-carboxylate;
tert-butyl 4-cyano-5-[(cyclopentylcarbonyl)amino]-3-methylthiophene-2-carboxylate;
tert-butyl 4-cyano-5-[(cyclohexylcarbonyl)amino]-3-methylthiophene-2-carboxylate;
tert-butyl 4-cyano-5-(isobutyrylamino)-3-methylthiophene-2-carboxylate;
tert-butyl 4-cyano-5-[(2,2-dimethylpropanoyl)amino]-3-methylthiophene-2-carboxylate;
benzyl 4-cyano-5-[(2-ethylbutanoyl)amino]-3-methylthiophene-2-carboxylate;
2-chlorobenzyl 4-cyano-5-[(2-ethylbutanoyl)amino]-3-methylthiophene-2-carboxylate;
3-chlorobenzyl 4-cyano-5-[(2-ethylbutanoyl)amino]-3-methylthiophene-2-carboxylate;
4-chlorobenzyl 4-cyano-5-[(2-ethylbutanoyl)amino]-3-methylthiophene-2-carboxylate;
2-cyanobenzyl 4-cyano-5-[(2-ethylbutanoyl)amino]-3-methylthiophene-2-carboxylate;
3-cyanobenzyl 4-cyano-5-[(2-ethylbutanoyl)amino]-3-methylthiophene-2-carboxylate;
4-cyanobenzyl 4-cyano-5-[(2-ethylbutanoyl)amino]-3-methylthiophene-2-carboxylate;
2-naphthylmethyl 4-cyano-5-[(2-ethylbutanoyl)amino]-3-methylthiophene-2-carboxylate;
3-(trifluoromethyl)benzyl 4-cyano-5-[(2-ethylbutanoyl)amino]-3-methylthiophene-2-carboxylate;
N-benzyl-4-cyano-N-ethyl-5-[(2-ethylbutanoyl)amino]-3-methylthiophene-2-carboxamide;
4-cyano-N-cyclopentyl-5-[(2-ethylbutanoyl)amino]-N-(4-fluorobenzyl)-3-methylthiophene-2-carboxamide;
N-benzyl-4-cyano-5-[(2-ethylbutanoyl)amino]-N,3-dimethylthiophene-2-carboxamide;
4-cyano-5-[(2-ethylbutanoyl)amino]-N,N,3-trimethylthiophene-2-carboxamide;
N-benzyl-4-cyano-5-[(2-ethylbutanoyl)amino]-N-isopropyl-3-methylthiophene-2-carboxamide;
4-cyano-5-[(2-ethylbutanoyl)amino]-N-[1-(hydroxymethyl)-2,2-dimethylpropyl]-3-methyl-N-(2-naphthylmethyl)thiophene-2-carboxamide;
N-(tert-butyl)-4-cyano-5-[(2-ethylbutanoyl)amino]-3-methyl-N-(2-naphthylmethyl)thiophene-2-carboxamide;
4-cyano-5-[(2-ethylbutanoyl)amino]-3-methyl-N-(2-naphthylmethyl)-N-(1,2,2-trimethylpropyl)thiophene-2-carboxamide;
4-cyano-N-cyclopentyl-5-[(2-ethylbutanoyl)amino]-3-methyl-N-(2-naphthylmethyl)thiophene-2-carboxamide;
4-cyano-5-[(2-ethylbutanoyl)amino]-3-methyl-N-(2-naphthylmethyl)-N-(1,2,3,4-tetrahydronaphthalen-1-yl)thiophene-2-carboxamide;
N-(tert-butyl)-4-cyano-5-[(2-ethylbutanoyl)amino]-N-(4-fluorobenzyl)-3-methylthiophene-2-carboxamide;
4-cyano-5-[(2-ethylbutanoyl)amino]-N-(4-fluorobenzyl)-3-methyl-N-(1,2,2-trimethylpropyl)thiophene-2-carboxamide;
4-cyano-N-(2,4-dichlorobenzyl)-5-[(2-ethylbutanoyl)amino]-N-isopropyl-3-methylthiophene-2-carboxamide, and
N-{3-cyano-4-methyl-5-[(4-phenylpiperidin-1-yl)carbonyl]thien-2-yl}-2-ethylbutanamide, and the pharmaceutically acceptable salts and solvates of the compounds listed above.

14. A pharmaceutical composition which is comprised of a compound of formula I: 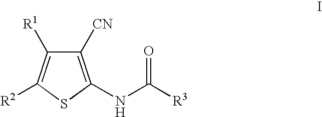
or a pharmaceutically acceptable salt or solvate thereof wherein:
R¹ is selected from the group consisting of: H, C₁₋₁₀alkyl, Aryl, Heteroaryl and Heterocyclyl,
said alkyl, Aryl, Heteroaryl and Heterocyclyl being optionally substituted with one to four substituents independently selected from R⁶;
R² is selected from the group consisting of: H, C₁₋₁₀ alkyl, C(O)C₁₋₁₀ alkyl, C(O)Aryl, C(O)Heteroaryl, C(O)Heterocyclyl CO₂R⁴ and C(O)NR⁴R⁵,
the alkyl, Aryl, Heteroaryl and Heterocyclyl portions of C(O)C₁₋₁₀alkyl, C(O)Aryl, C(O)Heteroaryl and C(O)Heterocyclyl being optionally substituted with one to four substituents independently selected from R⁶;
R³ is selected from the group consisting of: C₁₋₁₀alkyl and Aryl, said alkyl and Aryl being optionally substituted with one to four substituents independently selected from R⁶;
R⁴ is selected from the group consisting of: H, C₁₋₁₀alkyl, Aryl, Heteroaryl, Heterocyclyl, said alkyl, Aryl, Heteroaryl, and Heterocyclyl being optionally substituted with one to four substituents independently selected from R⁶;
R⁵ is selected from the group consisting of: C₁₋₁₀alkyl, Aryl, Heteroaryl and Heterocyclyl, said alkyl, cycloalkyl, Aryl Heteroaryl, and Heterocyclyl being optionally substituted with one to four substituents independently selected from R⁶;
when R² represents C(O)C₁₋₁₀alkyl, each R⁶ is independently selected from the group consisting of: halo, Aryl, Heteroaryl, Heterocyclyl, OR⁷, SR⁷, S(O)ₘR⁸, S(O)₂OR⁸, S(O)ₘNR⁷R⁸, NO₂, NR⁷R⁸, O(CR⁹R¹⁰)ₙNR⁷R⁸, C(O)R⁸, CO₂R⁷, CO₂(CR⁹R¹⁰)ₙCONR⁷R⁸, OC(O)R⁸, CN, C(O)NR⁷R⁸, NR⁷C(O)R⁸, OC(O)NR⁷R⁸, NR⁷C(O)OR⁸, NR⁷C(O)NR⁸R⁹, CR⁷(NOR⁸), (CR⁹R¹⁰)ₙ-Aryl, (CR⁹R¹⁰)ₙ-Heteroaryl, (CR⁹R¹⁰)ₙ-Heterocyclyl, CF₃ and OCF₃,
and when R² is C(O)Aryl, C(O)Heteroaryl or C(O)Heterocyclyl, and when R⁶ is a substituent on R³, R⁴ and R⁵, each R⁶ is independently selected from the group consisting of halo, C₁₋₇alkyl, Aryl, Heteroaryl, Heterocyclyl, OR⁷, SR⁷, S(O)ₘR⁸, S(O)₂OR⁸, S(O)ₘNR⁷R⁸, NO₂, NR⁷R⁸, O(CR⁹R¹⁰)ₙNR⁷R⁸, C(O)R⁸, CO₂R⁷, CO₂(CR⁹R¹⁰)ₙCONR⁷R⁸, OC(O)R⁸, CN, C(O)NR⁷R⁸, NR⁷C(O)R⁸, OC(O)NR⁷R⁸, NR⁷C(O)OR⁸, NR⁷C(O)NR⁸R⁹, CR⁷(NOR⁸), (CR⁹R¹⁰)ₙ-Aryl, (CR⁹R¹⁰)ₙ-Heteroaryl, (CR⁹R¹⁰)ₙ-Heterocyclyl, CF₃ and OCF₃;
wherein m is 0, 1 or 2 and n is an integer from 1 to 7, and the alkyl, Heterocyclyl, Aryl and Heteroaryl groups and portions are optionally substituted with 1-4 substituents selected from a group independently selected from R¹¹;
R⁷, R⁹ and R¹⁰ are independently selected from the group consisting of: H, C₁₋₇alkyl, Aryl, Ar-C₁₋₁₀alkyl and mono-, di- and tri- halo substituted Ar-C₁₋₁₀alkyl,
or one R⁹ and one R¹⁰ are taken together with the atoms to which they are attached and any intervening atoms and represent a ring of 3 to 8 members containing 0-2 heteroatoms independently selected from O, S and N;
R⁸ is selected from the group consisting of: C₁₋₁₀ alkyl, Aryl and C₁₋₁₀alkyl-Aryl; and
R¹¹ is selected from the group consisting of: halo, CN, C₁₋₄alkyl, Aryl, CF₃ and OH in combination with a pharmaceutically acceptable carrier.

15. A method of preventing or delaying the onset of type 2 diabetes mellitus in a mammalian patient in need thereof, comprising administering to said patient a compound of formula I: 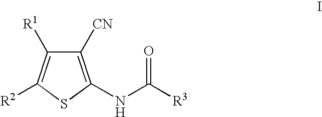
or a pharmaceutically acceptable salt or solvate thereof wherein:
R¹ is selected from the group consisting of: H, C₁₋₁₀alkyl, Aryl, Heteroaryl and Heterocyclyl,
said alkyl, Aryl, Heteroaryl and Heterocyclyl being optionally substituted with one to four substituents independently selected from R⁶;
R² is selected from the group consisting of: H, C₁₋₁₀ alkyl, C(O)C₁₋₁₀ alkyl, C(O)Aryl, C(O)Heteroaryl, C(O)Heterocyclyl CO₂R⁴ and C(O)NR⁴R⁵,
the alkyl, Aryl, Heteroaryl and Heterocyclyl portions of C(O)C₁₋₁₀alkyl, C(O)Aryl, C(O)Heteroaryl and C(O)Heterocyclyl being optionally substituted with one to four substituents independently selected from R⁶;
R³ is selected from the group consisting of: C₁₋₁₀alkyl and Aryl, said alkyl and Aryl being optionally substituted with one to four substituents independently selected from R⁶;
R⁴ is selected from the group consisting of: H, C₁₋₁₀alkyl, Aryl, Heteroaryl, Heterocyclyl, said alkyl, Aryl, Heteroaryl, and Heterocyclyl being optionally substituted with one to four substituents independently selected from R⁶;
R⁵ is selected from the group consisting of: C₁₋₁₀alkyl, Aryl, Heteroaryl and Heterocyclyl, said alkyl, cycloalkyl, Aryl Heteroaryl, and Heterocyclyl being optionally substituted with one to four substituents independently selected from R⁶;
when R² represents C(O)C₁₋₁₀alkyl, each R⁶ is independently selected from the group consisting of: halo, Aryl, Heteroaryl, Heterocyclyl, OR⁷, SR⁷, S(O)ₘR⁸, S(O)₂OR⁸, S(O)ₘNR⁷R⁸, NO₂, NR⁷R⁸, O(CR⁹R¹⁰)ₙNR⁷R⁸, C(O)R⁸, CO₂R⁷, CO₂(CR⁹R¹⁰)ₙCONR⁷R⁸, OC(O)R⁸, CN, C(O)NR⁷R⁸, NR⁷C(O)R⁸, OC(O)NR⁷R⁸, NR⁷C(O)OR⁸, NR⁷C(O)NR⁸R⁹, CR⁷(NOR⁸), (CR⁹R¹⁰)ₙ-Aryl, (CR⁹R¹⁰)ₙ-Heteroaryl, (CR⁹R¹⁰)ₙ-Heterocyclyl, CF₃ and OCF₃,
and when R² is C(O)Aryl, C(O)Heteroaryl or C(O)Heterocyclyl, and when R⁶ is a substituent on R³, R⁴ and R⁵, each R⁶ is independently selected from the group consisting of halo, C₁₋₇alkyl, Aryl, Heteroaryl, Heterocyclyl, OR⁷, SR⁷, S(O)ₘR⁸, S(O)₂OR⁸, S(O)ₘNR⁷R⁸, NO₂, NR⁷R⁸, O(CR⁹R¹⁰)ₙNR⁷R⁸, C(O)R⁸, CO₂R⁷, CO₂(CR⁹R¹⁰)ₙCONR⁷R⁸, OC(O)R⁸, CN, C(O)NR⁷R⁸, NR⁷C(O)R⁸, OC(O)NR⁷R⁸, NR⁷C(O)OR⁸, NR⁷C(O)NR⁸R⁹, CR⁷(NOR⁸), (CR⁹R¹⁰)ₙ-Aryl, (CR⁹R¹⁰)ₙ-Heteroaryl, (CR⁹R¹⁰)ₙ-Heterocyclyl, CF₃ and OCF₃;
wherein m is 0, 1 or 2 and n is an integer from 1 to 7, and the alkyl, Heterocyclyl, Aryl and Heteroaryl groups and portions are optionally substituted with 1-4 substituents selected from a group independently selected from R¹¹;
R⁷, R⁹ and R¹⁰ are independently selected from the group consisting of: H, C₁₋₇alkyl, Aryl, Ar-C₁₋₁₀alkyl and mono-, di- and tri- halo substituted Ar-C₁₋₁₀alkyl,
or one R⁹ and one R¹⁰ are taken together with the atoms to which they are attached and any intervening atoms and represent a ring of 3 to 8 members containing 0-2 heteroatoms independently selected from O, S and N;
R⁸ is selected from the group consisting of: C₁₋₁₀ alkyl, Aryl and C₁₋₁₀alkyl-Aryl; and
R¹¹ is selected from the group consisting of: halo, CN, C₁₋₄alkyl, Aryl, CF₃ and OH
said compound bein administered in an amount that is effective to prevent or delay the onset of type 2 diabetes mellitus.

16. A method of treating, preventing or delaying the onset of a disease or condition in a type 2 diabetes mellitus patient, said disease or condition being selected from the group consisting of: dyslipidemia selected from elevated serum cholesterol, elevated serum triglycerides, elevated serum low density lipoproteins and low levels of serum high density lipoprotein, microvascular or macrovascular changes and the sequellae of such conditions selected from coronary heart disease, stroke, peripheral vascular disease, hypertension, renal hypertension, nephropathy, neuropathy and retinopathy,
said method comprising administering to the type 2 diabetic patient a compound of formula I: 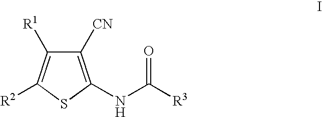
or a pharmaceutically acceptable salt or solvate thereof wherein:
R¹ is selected from the group consisting of: H, C₁₋₁₀alkyl, Aryl, Heteroaryl and Heterocyclyl,
said alkyl, Aryl, Heteroaryl and Heterocyclyl being optionally substituted with one to four substituents independently selected from R⁶;
R² is selected from the group consisting of: H, C₁₋₁₀ alkyl, C(O)C₁₋₁₀ alkyl, C(O)Aryl, C(O)Heteroaryl, C(O)Heterocyclyl CO₂R⁴ and C(O)NR⁴R⁵,
the alkyl, Aryl, Heteroaryl and Heterocyclyl portions of C(O)C₁₋₁₀alkyl, C(O)Aryl, C(O)Heteroaryl and C(O)Heterocyclyl being optionally substituted with one to four substituents independently selected from R⁶;
R³ is selected from the group consisting of: C₁₋₁₀alkyl and Aryl, said alkyl and Aryl being optionally substituted with one to four substituents independently selected from R⁶;
R⁴ is selected from the group consisting of: H, C₁₋₁₀alkyl, Aryl, Heteroaryl, Heterocyclyl, said alkyl, Aryl, Heteroaryl, and Heterocyclyl being optionally substituted with one to four substituents independently selected from R⁶;
R⁵ is selected from the group consisting of: C₁₋₁₀alkyl, Aryl, Heteroaryl and Heterocyclyl, said alkyl, cycloalkyl, Aryl Heteroaryl, and Heterocyclyl being optionally substituted with one to four substituents independently selected from R⁶;
when R² represents C(O)C₁₋₁₀alkyl, each R⁶ is independently selected from the group consisting of: halo, Aryl, Heteroaryl, Heterocyclyl, OR⁷, SR⁷, S(O)ₘR⁸, S(O)₂OR⁸, S(O)ₘNR⁷R⁸, NO₂, NR⁷R⁸, O(CR⁹R¹⁰)ₙNR⁷R⁸, C(O)R⁸, CO₂R⁷, CO₂(CR⁹R¹⁰)ₙCONR⁷R⁸, OC(O)R⁸, CN, C(O)NR⁷R⁸, NR⁷C(O)R⁸, OC(O)NR⁷R⁸, NR⁷C(O)OR⁸, NR⁷C(O)NR⁸R⁹, CR⁷(NOR⁸), (CR⁹R¹⁰)ₙ-Aryl, (CR⁹R¹⁰)ₙ-Heteroaryl, (CR⁹R¹⁰)ₙ-Heterocyclyl, CF₃ and OCF₃,
and when R² is C(O)Aryl, C(O)Heteroaryl or C(O)Heterocyclyl, and when R⁶ is a substituent on R³, R⁴ and R⁵, each R⁶ is independently selected from the group consisting of halo, C₁₋₇alkyl, Aryl, Heteroaryl, Heterocyclyl, OR⁷, SR⁷, S(O)ₘR⁸, S(O)₂OR⁸, S(O)ₘNR⁷R⁸, NO₂, NR⁷R⁸, O(CR⁹R¹⁰)ₙNR⁷R⁸, C(O)R⁸, CO₂R⁷, CO₂(CR⁹R¹⁰)ₙCONR⁷R⁸, OC(O)R⁸, CN, C(O)NR⁷R⁸, NR⁷C(O)R⁸, OC(O)NR⁷R⁸, NR⁷C(O)OR⁸, NR⁷C(O)NR⁸R⁹, CR⁷(NOR⁸), (CR⁹R¹⁰)ₙ-Aryl, (CR⁹R¹⁰)ₙ-Heteroaryl, (CR⁹R¹⁰)ₙ-Heterocyclyl, CF₃ and OCF₃;
wherein m is 0, 1 or 2 and n is an integer from 1 to 7, and the alkyl, Heterocyclyl, Aryl and Heteroaryl groups and portions are optionally substituted with 1-4 substituents selected from a group independently selected from R¹¹;
R⁷, R⁹ and R¹⁰ are independently selected from the group consisting of: H, C₁₋₇alkyl, Aryl, Ar-C₁₋₁₀alkyl and mono-, di- and tri- halo substituted Ar-C₁₋₁₀alkyl,
or one R⁹ and one R¹⁰ are taken together with the atoms to which they are attached and any intervening atoms and represent a ring of 3 to 8 members containing 0-2 heteroatoms independently selected from O, S and N;
R⁸ is selected from the group consisting of: C₁₋₁₀ alkyl, Aryl and C₁₋₁₀alkyl-Aryl; and
R¹¹ is selected from the group consisting of: halo, CN, C₁₋₄alkyl, Aryl, CF₃ and OH.
said compound being administered in an amount that is effective for treating, preventing or delaying the onset of such disease or condition.

17. A compound selected from the group consisting of:
tert-butyl 4-cyano-5-[(2-ethylbutanoyl)amino]-3-methylthiophene-2-carboxylate;
tert-butyl 4-cyano-5-[(cyclobutylcarbonyl)amino]-3-methylthiophene-2-carboxylate;
tert-butyl 4-cyano-5-[(cyclopentylcarbonyl)amino]-3-methylthiophene-2-carboxylate;
tert-butyl 4-cyano-5-[(cyclohexylcarbonyl)amino]-3-methylthiophene-2-carboxylate;
tert-butyl 4-cyano-5-(isobutyrylamino)-3-methylthiophene-2-carboxylate;
tert-butyl 4-cyano-5-[(2,2-dimethylpropanoyl)amino]-3-methylthiophene-2-carboxylate;
benzyl 4-cyano-5-[(2-ethylbutanoyl)amino]-3-methylthiophene-2-carboxylate;
2-chlorobenzyl 4-cyano-5-[(2-ethylbutanoyl)amino]-3-methylthiophene-2-carboxylate;
3-chlorobenzyl 4-cyano-5-[(2-ethylbutanoyl)amino]-3-methylthiophene-2-carboxylate;
4-chlorobenzyl 4-cyano-5-[(2-ethylbutanoyl)amino]-3-methylthiophene-2-carboxylate;
2-cyanobenzyl 4-cyano-5-[(2-ethylbutanoyl)amino]-3-methylthiophene-2-carboxylate;
3-cyanobenzyl 4-cyano-5-[(2-ethylbutanoyl)amino]-3-methylthiophene-2-carboxylate;
4-cyanobenzyl 4-cyano-5-[(2-ethylbutanoyl)amino]-3-methylthiophene-2-carboxylate;
2-naphthylmethyl 4-cyano-5-[(2-ethylbutanoyl)amino]-3-methylthiophene-2-carboxylate;
3-(trifluoromethyl)benzyl 4-cyano-5-[(2-ethylbutanoyl)amino]-3-methylthiophene-2-carboxylate;
N-benzyl-4-cyano-N-ethyl-5-[(2-ethylbutanoyl)amino]-3-methylthiophene-2-carboxamide;
4-cyano-N-cyclopentyl-5-[(2-ethylbutanoyl)amino]-N-(4-fluorobenzyl)-3-methylthiophene-2-carboxamide;
N-benzyl-4-cyano-5-[(2-ethylbutanoyl)amino]-N,3-dimethylthiophene-2-carboxamide;
4-cyano-5-[(2-ethylbutanoyl)amino]-N,N,3-trimethylthiophene-2-carboxamide;
N-benzyl-4-cyano-5-[(2-ethylbutanoyl)amino]-N-isopropyl-3-methylthiophene-2-carboxamide;
4-cyano-5-[(2-ethylbutanoyl)amino]-N-[1-(hydroxymethyl)-2,2-dimethylpropyl]-3-methyl-N-(2-naphthylmethyl)thiophene-2-carboxamide;
N-(tert-butyl)-4-cyano-5-[(2-ethylbutanoyl)amino]-3-methyl-N-(2-naphthylmethyl)thiophene-2-carboxamide;
4-cyano-5-[(2-ethylbutanoyl)amino]-3-methyl-N-(2-naphthylmethyl)-N-(1,2,2-trimethylpropyl)thiophene-2-carboxamide;
4-cyano-N-cyclopentyl-5-[(2-ethylbutanoyl)amino]-3-methyl-N-(2-naphthylmethyl)thiophene-2-carboxamide;
4-cyano-5-[(2-ethylbutanoyl)amino]-3-methyl-N-(2-naphthylmethyl)-N-(1,2,3,4-tetrahydronaphthalen-1-yl)thiophene-2-carboxamide;
N-(tert-butyl)-4-cyano-5-[(2-ethylbutanoyl)amino]-N-(4-fluorobenzyl)-3-methylthiophene-2-carboxamide;
4-cyano-5-[(2-ethylbutanoyl)amino]-N-(4-fluorobenzyl)-3-methyl-N-(1,2,2-trimethylpropyl)thiophene-2-carboxamide;
4-cyano-N-(2,4-dichlorobenzyl)-5-[(2-ethylbutanoyl)amino]-N-isopropyl-3-methylthiophene-2-carboxamide, and
N-{3-cyano-4-methyl-5-[(4-phenylpiperidin-1-yl)carbonyl]thien-2-yl}-2-ethylbutanamide, and the pharmaceutically acceptable salts and solvates of the compounds listed above.
